(19) United States
(12) Patent Application Publication
(10) Pub. No.: US 2017/0231782 A1
(43) Pub. Date: Aug. 17, 2017

(54) SPINAL FUSION SPACER WITH INTEGRATED GRAFT DELIVERY

(71) Applicant: MI4SPINE, LLC, Bloomfield Village, MI (US)

(72) Inventors: MIGUELANGELO J. PEREZ-CRUET, BLOOMFIELD, MI (US); JOHN R. PEPPER, CHESHIRE, CT (US)

(21) Appl. No.: 15/043,396

(22) Filed: Feb. 12, 2016

(51) Int. Cl. A61F 2/46; A61F 2/44

(52) U.S. Cl. CPC A61F 2/4601; A61F 2/447; A61F 2/4611; A61F 2002/4475; A61F 2002/4629; A61F 2002/4623

(57) ABSTRACT

A spinal fusion implant that is inserted into a cleared disc space during a spinal fusion surgical procedure using an insertion tool, where the insertion tool also provides delivery of bone graft material once the implant is in the disc space. The implant includes a perimeter portion having a front wall, a back wall and opposing side walls defining an enclosure, where the perimeter portion is open in an up and down direction, and where the front wall includes an opening extending therethrough to the enclosure and being configured to accept the bone graft material to fill the enclosure.

### BACKGROUND OF THE INVENTION

Field of the Invention

This invention relates generally to a spinal fusion implant and, more particularly, to a spinal fusion implant that is inserted between two adjacent vertebra in a cleared disc space using an insertion tool, and is then filled with bone graft material through the tool once the implant is provided within the disc space.

Discussion of the Related Art

The human spine includes a series of vertebrae interconnected by connective tissue referred to as discs that act as a cushion between the vertebrae. The discs allow for movement of the vertebrae so that the back can bend and rotate.

Spinal fusion is a surgical procedure that fuses two or more vertebrae together using bone grafts and/or other devices. Spinal fusion is a commonly performed procedure for the treatment of chronic neck and back pain refractory to non-operative treatments. Spinal fusion is used to stabilize or eliminate motion of vertebrae segments that may be unstable, i.e., move in an abnormal way, that may lead to pain and discomfort. Spinal fusion is typically performed to treat injuries to the vertebrae, degeneration of the spinal discs, abnormal spinal curvature and a weak or unstable spine.

Spinal fusion generally requires a graft material, usually bone material, to fuse the vertebrae together. The bone graft material can be placed over the spine to fuse adjacent vertebrae together. Alternatively, a cage is positioned between the vertebrae being fused, and is filed with the bone graft material. The cage can include holes that allow the vertebrae and the graft material to grow together to provide the fusion. The cage supports the weight of adjacent vertebrae while the fusion is occurring through the holes in the cage.

Typically the bone graft material is autogenous bone material taken from the patient, or allograft bone material harvested from a cadaver. Synthetic bone material can also be used as the graft material. Generally, the patient's own bone material offers the best fusion material and is the current “gold standard.” Known bone fusion materials include an iliac crest harvest from the patient, bone graft extenders, such as hydroxyapetite and demineralized bone matrix, and bone morphogenic protein.

In an attempt to preserve normal anatomical structures during spinal surgery, minimally invasive surgical procedures have been devised. One such procedure involves the use of a series of muscle dilators that separate the muscle fibers of the spine to create a pathway to the spine. A Kirschner (K-wire) is initially introduced through a small incision and directed towards the spinal pathology. The position of the K-wire is visualized by a fluoroscopic imaging system to identify its location. An initial narrow diameter muscle dilator is passed over the K-wire, and the K-wire is removed and subsequent larger muscle dilators are continually passed. When the opening is large enough, an access tube or retractor is positioned around the last muscle dilator through which the surgery is performed. The inner sequential muscle dilators are then removed allowing the surgeon to operate through the tubular retractor. The retractors come in a variety of lengths and diameters for different patients and procedures.

As mentioned above, a cage is typically positioned in the interbody region between the vertebrae after the disc has been removed, where the cage typically has a box-like design. The cage is forced into the interbody region through the surgical area where the bone and disc have been removed. The cage is filled with the bone graft material that subsequently fuses the vertebrae together. However, the known cage designs require that the bone graft material be placed in the cage prior to the cage being inserted into the interbody region, which often results in loss of the graft material during insertion of the cage, and thus limits the amount of bone material placed in the disc space. Also, once the cages are placed, they are difficult to remove and reposition.

### SUMMARY OF THE INVENTION

The present disclosure describes a spinal fusion implant that is inserted into a cleared disc space during a spinal fusion surgical procedure using an insertion tool, where the insertion tool also provides delivery of bone graft material once the implant is in the disc space. The implant includes a perimeter portion having a front wall, a back wall and opposing side walls defining an enclosure, where the perimeter portion is open in an up and down direction, and where the front wall includes an opening extending therethrough to the enclosure and being configured to accept the bone graft material to fill the enclosure.

Additional features of the present invention will become apparent from the following description and appended claims, taken in conjunction with the accompanying drawings.

### BRIEF DESCRIPTION OF THE DRAWINGS

FIG. 1 is an isometric view of a spinal fusion assembly including an implant, an insertion tube and a drive tool;

FIG. 2 is a cut-away, cross-sectional view of the spinal fusion assembly;

FIG. 3 is an isometric view of the implant shown in FIGS. 1 and 2;

FIG. 4 is a top view of the implant shown in FIG. 3;

FIG. 5 is a back view of the implant shown in FIG. 3;

FIG. 6 is a cross-sectional view of the implant shown in FIG. 3;

FIG. 7 is an isometric view of the insertion tool shown in FIG. 1 including a threaded tip;

FIG. 8 is an isometric view of the drive tool shown in FIG. 1;

FIG. 9 is an isometric view of the implant shown in FIG. 3 including a threaded plug inserted therein;

FIG. 10 is an illustration of opposing vertebrae showing the implant therebetween;

FIG. 11 is an isometric view of another implant;

FIG. 12 is an isometric view of a cover plate for the implant shown in FIG. 11;

FIG. 13 is an isometric view of an insertion tool including a pincher for inserting the implant shown in FIG. 11;

FIG. 14 is an isometric view of another implant suitable for a TLIF surgical procedure; and

FIG. 15 is an isometric view of another implant suitable for corpectomy surgical procedure.

### DETAILED DESCRIPTION OF THE EMBODIMENTS

The following discussion of the embodiments of the invention directed to spinal fusion implants is merely exemplary in nature, and is in no way intended to limit the invention or its applications or uses.

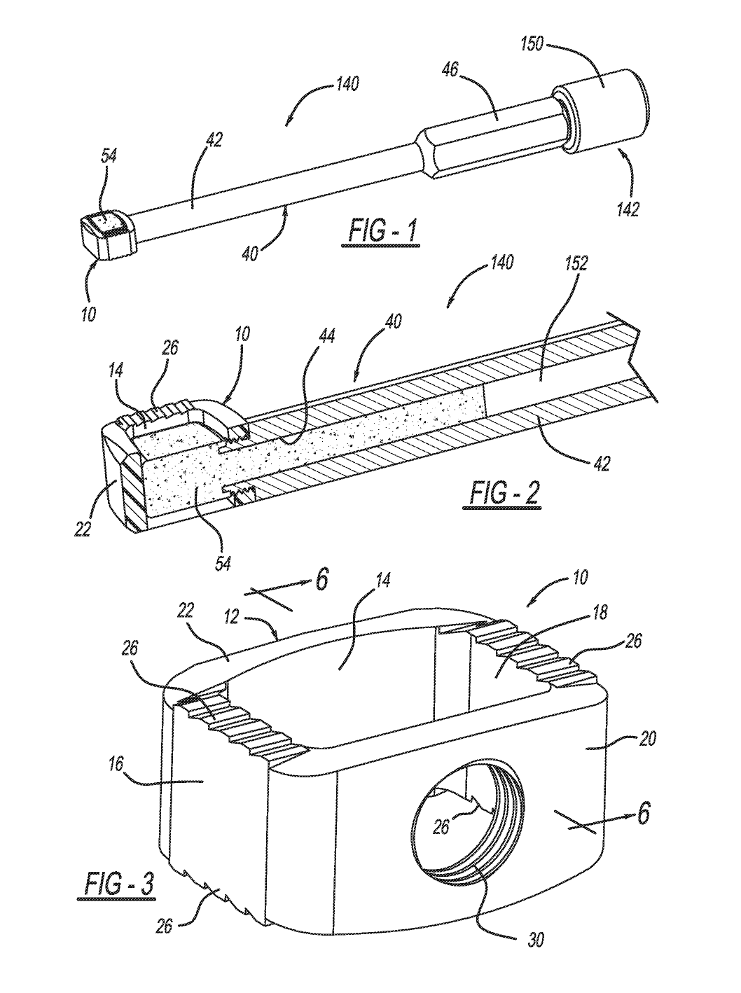

FIG. 1 is an isometric view and FIG. 2 is a cut-away, cross-sectional view of a spinal fusion assembly 140 including a spinal fusion implant 10, an insertion tool 40 and a drive tool 142. As will be discussed in detail below, the insertion tool 40 positions the implant 10 between adjacent vertebrae, such as in the lumbar spine, after the disc space has been cleared therebetween during a spinal fusion surgical procedure, and especially a minimally invasive spinal surgical procedure, and bone graft material 54 is delivered to the implant 10 once the implant 10 is appropriately placed by forcing the bone graft material 54 down a bore in the insertion tool 40 using the drive tool 142.

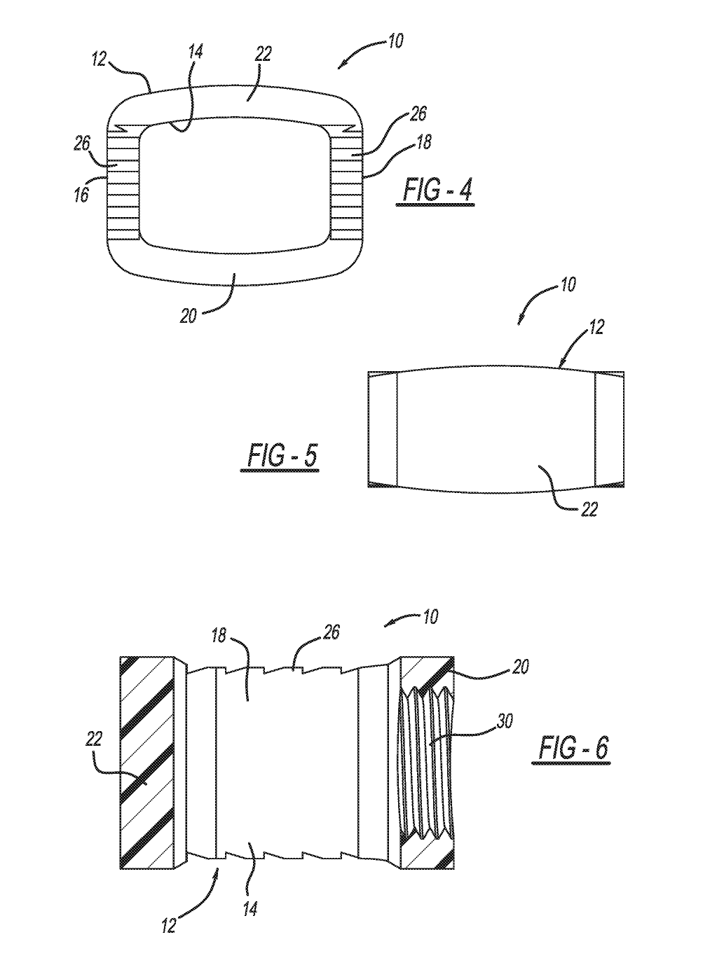

FIG. 3 is an isometric view, FIG. 4 is a top view, FIG. 5 is a back view and FIG. 6 is a cross-sectional view of the implant 10 having an outer perimeter portion 12 defining an enclosure 14 therein. The implant 10 can be provided in different sizes to accommodate the anatomy of different patients and so that it can be relatively easily slipped into the disc space through a dilator tube (not shown) used in minimally invasive surgical procedures without risk of injury to the nerve roots through the same channel that the disc has been removed from. In this embodiment, the perimeter portion 12 is generally square, however, other shapes may be applicable. Further, the implant 10 is a single piece member and is made of a suitable surgical material, such as titanium, PEEK, etc.

The perimeter portion 12 includes opposing side walls 16 and 18 and opposing front and back walls 20 and 22 such that the implant 10 is open to the top and bottom, as shown. Top and bottom surfaces of the side walls 16 and 18 include ridges 26 that allow the implant 10 to better grab the vertebral bodies. A threaded opening 30 is provided through the front wall 20 that accepts the insertion tool so that the implant 10 can be positioned in the disc space and through which bone graft material can be delivered to the enclosure 14. The opening 30 could be in other walls of the implant 10 for other surgical procedures.

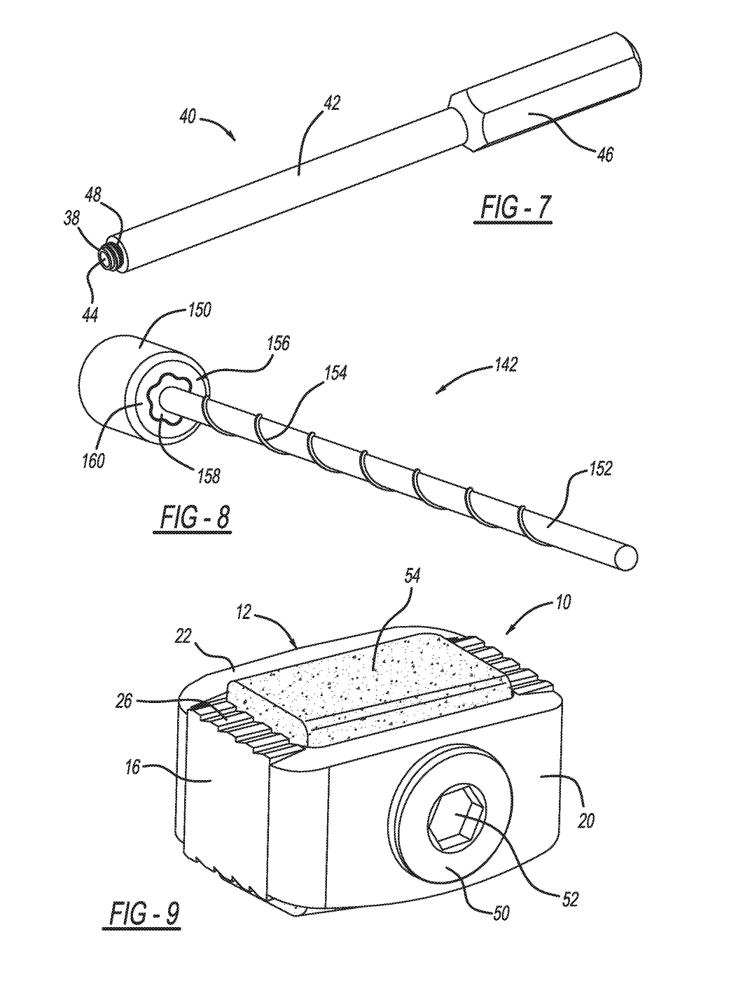

FIG. 7 is an isometric view of the insertion tool 40 that is a non-limiting example of a device that can position the implant 10 between the vertebra during the surgical procedure. The tool 40 includes an elongated shaft 42 having an internal bore 44 extending therethrough, and includes a handle portion 46 at one end and a threaded tip 48 having a rim 38 at an opposite end. The threaded tip 48 is threaded into the opening 30 when the surgeon is ready to insert the implant 10. Once the implant 10 is inserted into the disc space, the surgeon will deliver bone graft material through the bore 44 and into the enclosure 14 in any suitable manner. The surgeon will then unthread the tool 40 from the implant 10 to release it therefrom.

FIG. 8 is an isometric view of the drive tool 142 that includes a knob 150 with a rod 152 extending therefrom, where a helical screw 154 is wound around the rod 152. The pitch of the screw 154 is merely for illustrative purposes, where other pitches may be more practical. A clutch mechanism 156 is provided within the knob 150 and includes as one representative example an inner portion 158 rigidly secured to the rod 152 and an outer portion 160 rotatably mounted to the inner portion 158 under enough force. Other types of clutch mechanisms may also be applicable. The rod 152 is inserted in the bore 44 along with the bone graft material 54 and is rotated by the knob 150 to drive the bone graft material 54 into the enclosure 14, where the clutch mechanism 156 prevents to much rotational force that may drive the bone graft material 54 out of the enclosure 14. It is noted that other types and configurations of drive tools can be employed.

Once the tool 40 is removed from the implant 10, a plug 50 is then threaded into the opening 30, as shown in FIG. 9, where the plug 50 includes a hexagonal-shaped indentation 52 that accepts a wrench (not shown) to turn the plug 50. Also shown in FIG. 9 is the bone graft material 54 in the enclosure 14. By providing the bone graft material 54 in the enclosure 14 after the implant 10 is inserted, more bone graft material is able to be provided in the disc space, thus increasing the integrity of the fusion over the known procedures of filling the implant with the bone graft material before it is inserted into the disc space.

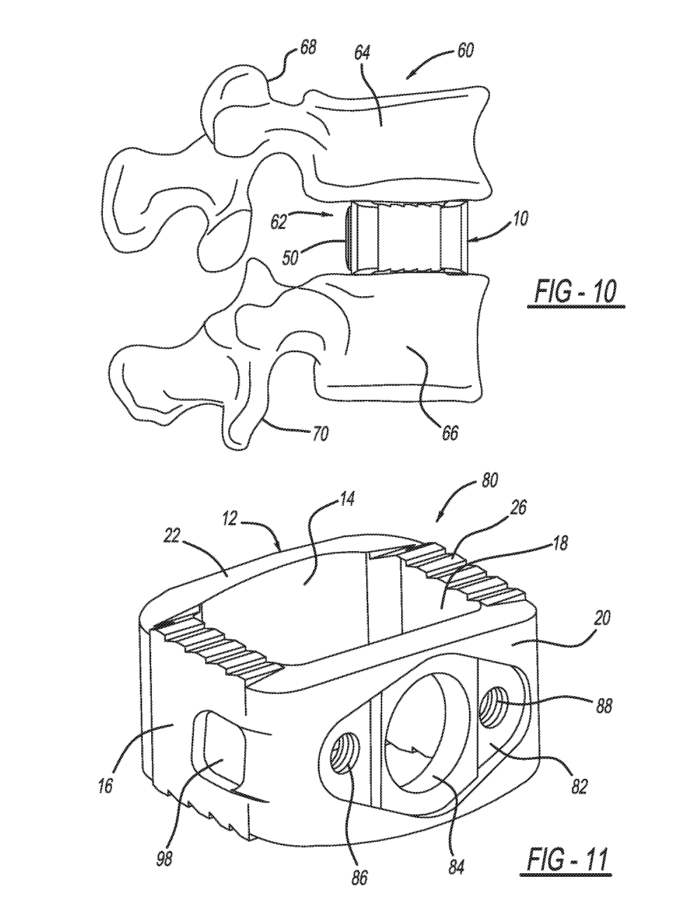

FIG. 10 is an illustration 60 showing the implant 10 positioned in a cleared disc space 62 between two vertebral bodies 64 and 66 of two adjacent vertebrae 68 and 70, respectively, as discussed above.

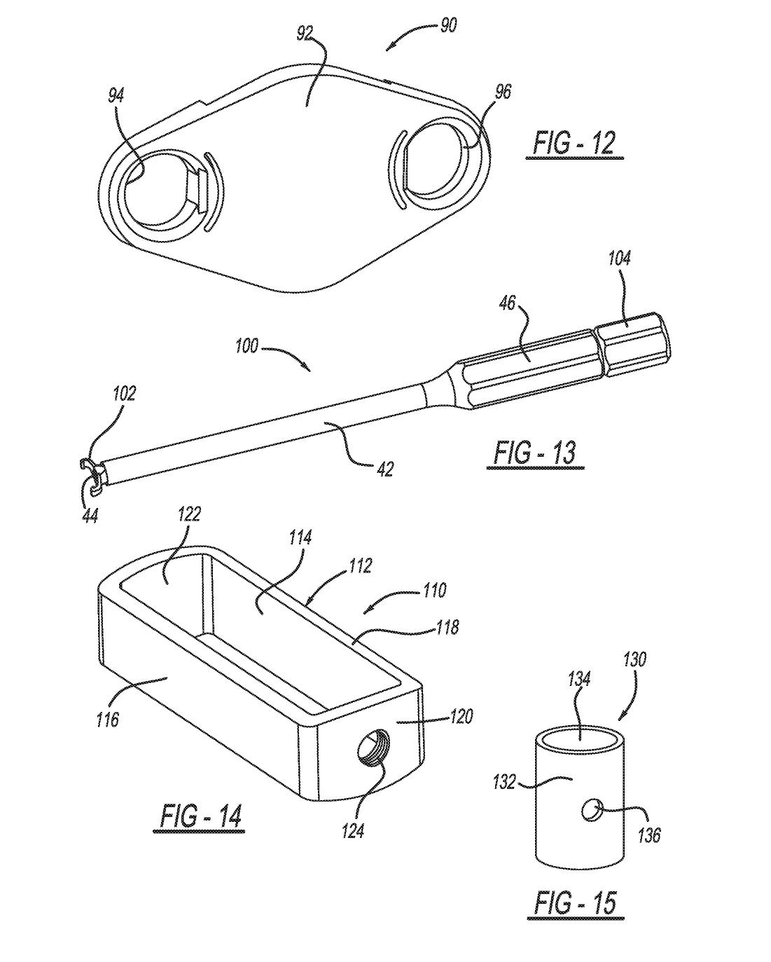

FIG. 11 is an isometric view of an implant 80 similar to the implant 10, where like elements are identified by the same reference number. In this design, a recessed section 82 is provided within the front wall 20, where a circular opening 84 is provided in the recessed section 82 through which the bone graft material can be delivered to the enclosure 14. Once the enclosure 14 is filled with the bone graft material 54, the opening 84 is closed off with a cover plate 90 shown in FIG. 12 that includes a front face 92 and has the same shape as the recessed section 82. The implant 80 includes two threaded openings 86 and 88 on opposite sides of the opening 84 and the cover plate 90 includes two openings 94 and 96 that line up with the openings 86 and 88, respectively, when the cover plate 90 is positioned within the recessed section 82. Two bolts (not shown) are then inserted through the openings 94 and 96 and threaded into the openings 86 and 88 to secure the cover plate 90 to the implant 80 and close off the opening 84 after the bone graft material 54 is delivered to the enclosure 14.

In this embodiment, the implant 80 is inserted into the disc space 62 in a different manner than the implant 10. Particularly, the side wall 16 includes a recess 98 and the side wall 18 includes an identical recess (not shown) that are configured to receive an insertion tool. FIG. 13 is an isometric view of an insertion tool 100 that is similar to the insertion tool 40, where like elements are identified by the same reference number, for inserting the implant 80. Instead of the threaded tip 48, the tool 100 includes a pincher 102 that is configured to engage the recesses 98. The pincher 102 can be opened and closed in a known manner, such as by rotating a control knob 104 provided adjacent to the handle portion 46. Thus, the tool 100 can be locked to the implant 80 for inserting it into the disc space 62. The implant 80 is then filled with the bone graft material through the bore 44 and the opening 84. The knob 104 is then turned to release the pincher 102 from the recesses 98 once the implant has been filled, and the opening 84 is then covered with the cover plate 90.

As mentioned, the implant can come in a variety of shapes and sizes. To illustrate this, FIG. 14 is an isometric view of an implant 110 having an elongated perimeter portion 112 defining an enclosure 114 therein, where the implant 110 may have the proper size for an XLIF surgical procedure. The perimeter portion 112 includes opposing side walls 116 and 118 and opposing front and back walls 120 and 122 such that the implant 14 is open to the top and bottom, as shown. A threaded opening 124 is provided through the front wall 120 that accepts the insertion tool 40 so that the implant 110 can be positioned in the disc space 62 and through which bone graft material can be delivered to the enclosure 114.

FIG. 15 is an isometric view of an implant 130 having an elongated tubular body 132 with a general oval shape and defining an enclosure 134 therein that may have application for a corpectomy procedure where the vertebral body of a vertebra has been removed. The body 132 includes an opening 136 through which bone graft material can be delivered to the enclosure 134 in the manner discussed above. The opening 136 is then closed off using a plug, such as discussed above.

The foregoing discussion discloses and describes merely exemplary embodiments of the present invention. One skilled in the art will readily recognize from such discussion and from the accompanying drawings and claims that various changes, modifications and variations can be made therein without departing from the spirit and scope of the invention as defined in the following claims.

What is claimed is:

1. An implant comprising a perimeter portion including a plurality of walls defining an enclosure, said perimeter portion being open in both an up and down direction, wherein one of the walls includes an opening extending therethrough to the enclosure and being configured to accept a bone graft material to fill the enclosure after the implant is inserted into a cleared disc space between two opposing vertebral bodies.

2. The implant according to claim 1 wherein the implant is configured to be removable secured to an insertion tool that inserts the implant into the disc space.

3. The implant according to claim 2 wherein the insertion tool includes a bore through which the bone graft material is delivered to the enclosure.

4. The implant according to claim 3 wherein the bone graft material is driven down the bore with a drive tool.

5. The implant according to claim 4 wherein the drive tool includes a helical screw that drives the bone graft material down the bore upon rotation of the drive tool.

6. The implant according to claim 5 wherein the drive tool includes a clutch mechanism that prevents the drive tool from being rotated above a predetermined force.

7. The implant according to claim 2 wherein the opening is a threaded opening that is configured to accept the insertion tool in a threaded engagement.

8. The implant according to claim 2 wherein the plurality of walls include opposing side walls each having a recess and the insertion tool includes a pincher that is operable to engage the recesses.

9. The implant according to claim 1 further comprising a plug inserted into the opening after the bone graft material is delivered to the enclosure.

10. The implant according to claim 1 further comprising a cover plate that is mounted to the one wall to cover the opening after the bone graft material is delivered to the enclosure.

11. The implant according to claim 10 wherein the cover plate fits within a shaped recessed section in the one wall.

12. The implant according to claim 1 wherein ridges are formed in upper and lower surfaces of opposing side walls.

13. The implant according to claim 1 wherein the perimeter portion is generally square.

14. An implant comprising a perimeter portion including a front wall, a back wall and opposing side walls defining an enclosure, said perimeter portion being open in both an up and down direction, wherein ridges are formed in upper and lower surfaces of the opposing side walls, said front wall including a threaded opening extending therethrough to the enclosure and being configured to accept a bone graft material to fill the enclosure after the implant is inserted into a cleared disc space between two opposing vertebral bodies, wherein the implant is configured to be removable secured to an insertion tool that inserts the implant into the disc space, said insertion tool including a bore through which the bone graft material is delivered to the enclosure, wherein the opening accepts the insertion tool in a threaded engagement, and wherein a plug is inserted into the opening after the bone graft material is delivered to the enclosure.

15. A method for surgically implanting an implant into a cleared disc space between two opposing vertebral bodies, said method comprising:
coupling the implant to an insertion tool, said insertion tool including a bore;
inserting the implant into the disc space using the insertion tool;
delivering bone graft material to an enclosure within the implant through the bore in the insertion tool and an opening in a wall of the implant using a drive tool;
removing the insertion tool from the implant; and
covering the opening.

16. The method according to claim 15 wherein coupling the implant to the insertion tool includes threading a threaded tip on the insertion tool into the opening.

17. The method according to claim 15 wherein coupling the implant to the insertion tool includes grasping recesses in the perimeter portion with a pincher on the insertion tool.

18. The method according to claim 15 wherein delivering the bone graft material includes rotating the drive tool so that a helical screw on the drive tool drives the bone graft material down the bore.

19. The method according to claim 18 wherein the drive tool includes a clutch mechanism that prevents the drive tool from being rotated above a predetermined force.

20. The method according to claim 15 wherein covering the opening includes threading a plug into the opening.

21. The method according to claim 15 wherein covering the opening includes mounting a cover plate to the front wall to cover the opening.

22. The method according to claim 21 wherein the cover plate is secured within a recessed section in the wall.

23. The method according to claim 15 wherein the implant includes a perimeter portion having a front wall, a back wall and opposing side walls defining the enclosure, said perimeter portion being open in an up and down direction.
